(19) United States
(12) Patent Application Publication
(10) Pub. No.: US 2008/0228247 A1
(43) Pub. Date: Sep. 18, 2008

(54) Health Care and Physical Therapy Device For Gathering Energy

(76) Inventor: Kam Moon Fung, Hong Kong (CN)

Correspondence Address: JOHN CHEN, ROOM 303, 3F, NO. 25, SEC. 1, CHANGAN E. ROAD, TAIPEI, 10441, TW

(21) Appl. No.: 11/816,529

(22) PCT Filed: Dec. 22, 2005
(86) PCT No.: PCT/CN2005/002286

§ 371 Date: Apr. 18, 2008

(30) Foreign Application Priority Data
Feb. 18, 2005 (CN) 200520054939.2

(51) Int. Cl. A61F 7/00

(52) U.S. Cl. 607/107

(57) ABSTRACT

The present invention provides a healthy care device comprising a casing having an inlet and an outlet on two opposite side thereof; a heater and a fan installed inside the casing; an air guiding sleeve having one end connected to the outlet of the casing and another end with contact area to a patient's skin, and an energy concentrating element installed on the air guiding sleeve. An exhaust opening is installed in the air guiding sleeve. The air induced by the fan is heated by the heater, and runs through the outlet to the air guiding sleeve to reach to the patient's skin surface. And the exhaust opening is applied to exhaust the hot air. With convective air, modulated temperature and adjustable air flow, the hot air gradually warms up patient's skin without burning the patient. The safety and the reliability are reached. For various applications, energy wave coating can be applied on energy concentrating element. The energy concentrating element, blown through by hot air, can generate infrared, magnetic energy, thermal energy, etc. The various kinds of energy can be focused to radiate the energy wave to enhance metabolism, to improve blood and various circulatory systems, and to balance immunity for beneficial therapeutic results. In addition, the temperature sensor and temperature control circuit ensure the temperature not to be overheating. Thus, it makes the application to be safe and reliable.

### BACKGROUND OF THE PRESENT INVENTION

1. Field of Invention

The present invention relates to a health care and physical therapy device for gathering energy, more particularly to a health care device by heating process.

2. Description of Related Arts

Presently, the general local or international practice on reducing inflammation, relieving pain, or enhancing blood circulation are by medicine or physiotherapy. Generally, the physiotherapy is by the use of IR (infrared ray) or magnetic energy, which is good for reducing inflammation, relieving pain, or enhancing blood circulation. However, the therapeutic equipments inducing infrared and magnetic energy are complex and expensive, and the effect is not remarkable on the practical application. The conventional physiotherapy in China includes acupuncture, moxibustion, and surface hot pressing. Although these physiotherapies are more remarkable, they are heavily relying on operator's skill. With simple method yet complicated manual procedure, an unskilled operator could easily hurt patients by pricking or burning. Therefore, there's a need to be overcome the inconvenience and the risk.

### SUMMARY OF THE PRESENT INVENTION

An Object of the present invention is to provide a solution device to the un-safe condition for the conventional physiotherapy and is to provided a simple structure with gradual heating process on the affected area of a patient's body as a combining process based on Chinese and western methods.

The present invention is to use a device comprising: a casing, the casing having an inlet and an outlet on two opposite sides thereof; a fan, the fan installed inside the casing; a heater, the heater installed inside the casing; an air guiding sleeve, the air guiding sleeve having one end connected to the outlet of the casing and having another end to contact with patient's skin; an energy concentrating element, the energy concentrating element installed in the guiding sleeve.

A temperature sensor is installed on the air guiding sleeve near the contact area to a patient's skin; and a temperature control circuit is connecting to the temperature sensor, wherein the temperature control circuit connected to the heater and the fan, whereby the temperature sensor measures temperature to modulate the power of the fan and the heater.

The exhaust opening is installed on one place selected from the side wall of the air guiding sleeve, or on the connecting portion of the air guiding sleeve with the outlet of the casing.

The air guiding sleeve has at least one vent hole on the near side of the contact area to patient's skin.

The energy concentrating element is a circle structure made of the material selected the group of copper, silver, and carbon fiber.

The heater is made of selected from the group of heating wire, heating tube, and infrared tube.

The casing further comprises a handle for operation; and a switch on the handle.

The air guiding sleeve is further comprising an air guiding frame, and a plurality of vent holes are evenly spread on the surface of the guiding frame.

The air guiding sleeve is further comprising a frame structure for scraping, and a tube shape is placed on the frame structure for scraping.

On comparing with the prior art, the present invention of health care device applies the heater to heat up the air induced by the fan; the hot air runs through the outlet to the air guiding sleeve to reach to the patient's skin surface. And the exhaust opening is applied to exhaust the hot air. The hot air gradually warms up patient's skin without burning the patient. The safety and the reliability are reached. And the hot air can heat the energy concentrating element inside the air guiding sleeve to generate infrared, magnetic energy, thermal energy or ionic energy. The various kinds of energy can be focused to radiate the energy wave to enhance the body's self-curing ability and blood circulation. The combination of Chinese and western curing method is achieved. In addition, the unbalanced immunization system, internal organs and the circulation system in body can be improved. Also, a flashboard can be added on the air exhaust opening. The size of the air exhaust opening can be adjusted by switching the flashboard, which changes the amount of the air flow. Then it achieves the function of adjusting the temperature of the outlet.

One or part or all of these and other features and advantages of the present invention will become readily apparent to those skilled in this art from the following description wherein there is shown and described a preferred embodiment of this invention, simply by way of illustration of one of the modes best suited to carry out the invention. As it will be realized, the invention is capable of different embodiments, and its several details are capable of modifications in various, obvious aspects all without departing from the invention. Accordingly, the drawings and descriptions will be regarded as illustrative in nature and not as restrictive.

### BRIEF DESCRIPTION OF THE DRAWINGS

FIG. 1 illustrates an embodiment of a health care device as a cross-section view for the first structure of the present invention;

FIG. 2 illustrates an embodiment of a health care device as a cross-section view for the second structure of the present invention;

FIG. 3 illustrates an air guiding frame in the FIG. 2;

FIG. 4 illustrates a side view on A-direction in the FIG. 3;

FIG. 5 illustrates an embodiment of a frame structure for scraping, and the frame structure for scraping is instead of air guiding frame;

FIG. 6 illustrates a side view on B-direction in the FIG. 5;

FIG. 7 illustrates an embodiment of a health care device as a cross-section view for the third structure of the present invention; and

FIG. 8 illustrates an embodiment of a circuit diagram for FIGS. 1-2.

### DETAILED DESCRIPTION OF THE PREFERRED EMBODIMENTS

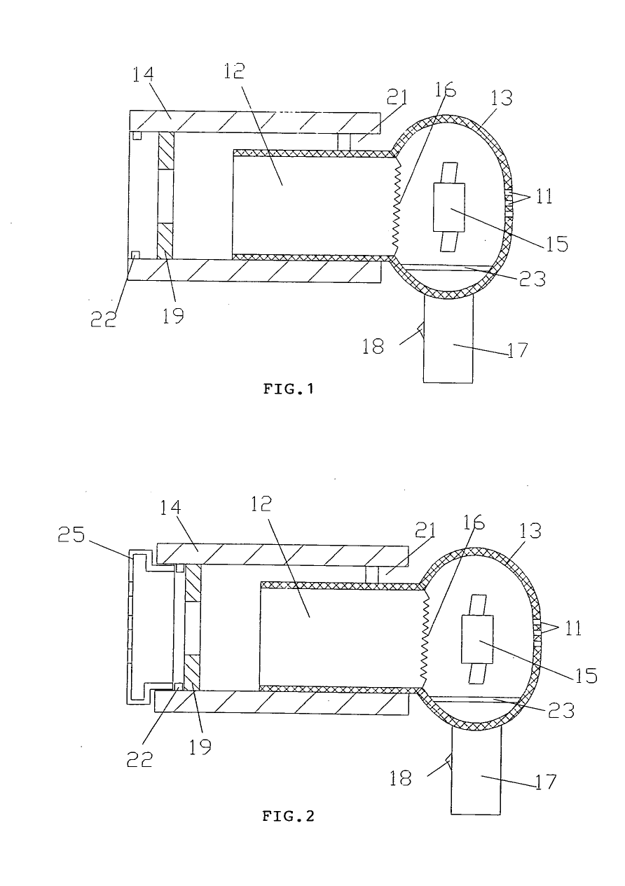

Referring to FIGS. 1-8, these are embodiments of the present invention. The below are the descriptions in detail.

For the described in the FIG. 1, this is a embodiment of the present invention, comprising: a casing 13, the casing having an inlet 11 and an outlet 12 on two opposite sides thereof; an air guiding sleeve 14, the air guiding sleeve 14 (with void space inside) having an exhaust opening 21 and connected to the outlet 12 of the casing 13. A fan 15 and a heater 16 are installed inside the casing 13, and the casing 13 can be a cylinder shape. Wherein the heater 16 is made of selected from the group of heating wire, heating tube, and infrared tube. The outside power source can provide power to the heater 16 and the fan 15. The casing 13 can further comprise a handle 17 for operation and a switch 18 installed on the handle 17. The fan 15 induces the outside air into the casing 13, and using the heating 16 to heat the air to exhaust to the outlet 12 to reach patient's skin.

An energy concentrating element 19 is installed in the guiding sleeve 14. The energy concentrating element 19 is a circle structure made of the material selected the group of copper, silver, and carbon fiber. The hot air can heat the energy concentrating element inside the air guiding sleeve to generate infrared, magnetic energy, hot energy or ionic energy. The various kinds of energy can be focused to radiate the energy wave to enhance the body's self-curing ability. In the embodiment the air guiding sleeve 14 is a void cylinder and installed on the outlet 12 of the casing 13. The air guiding sleeve 14 can have a plurality of vent holes 20 on the near side of the contact area to a patient's skin. The exhaust opening 21 is installed on one place selected from the side wall of the air guiding sleeve 14, and the connecting portion of the air guiding sleeve 14 with the outlet of the casing 13. The exhaust opening 21 is for hot air exhaust.

During the applied period, the air guiding sleeve 14 is contacting on the patient's skin. As the power is turned on, the fan and the heater are activated. The outside air is induced into the casing 13 and heated, and then runs through the outlet 12 and the air guiding sleeve 14 combining the air guiding frame 25 or the frame structure for scraping 26 to reach patient's skin. And the hot air is outgoing by the outlet 12 to get the gradually heating property on the air guiding sleeve 14. The patient's skin can easily adapt the gradually heated temperature which can enhance the lymph circulation and blood circulation; thus, it provides health benefits to patients with rheumatism or arthritis. Accordingly, the hot air can heat the energy concentrating element 19 inside the air guiding sleeve 14 to generate infrared, magnetic energy, thermal energy or ionic energy. The various kinds of energy can be focused to radiate the energy wave for health benefits.

For the safety concern, a temperature sensor 22 is installed on the air guiding sleeve 14 near the contact area to a patient's skin, and a temperature control circuit 23 (inside the casing 13) is connected to the temperature sensor 22. Wherein the temperature control circuit 23 is connected to the heater 16 and the fan 15, whereby the temperature sensor 22 measures the temperature inside the air guiding sleeve 14. When the measured temperature reaches the set temperature, the temperature control circuit 23 would modulate the power of the fan 15 and the heater 16. Then it is ensured that the temperature inside the air guiding sleeve is not overheated, preventing from burning patient's skin.

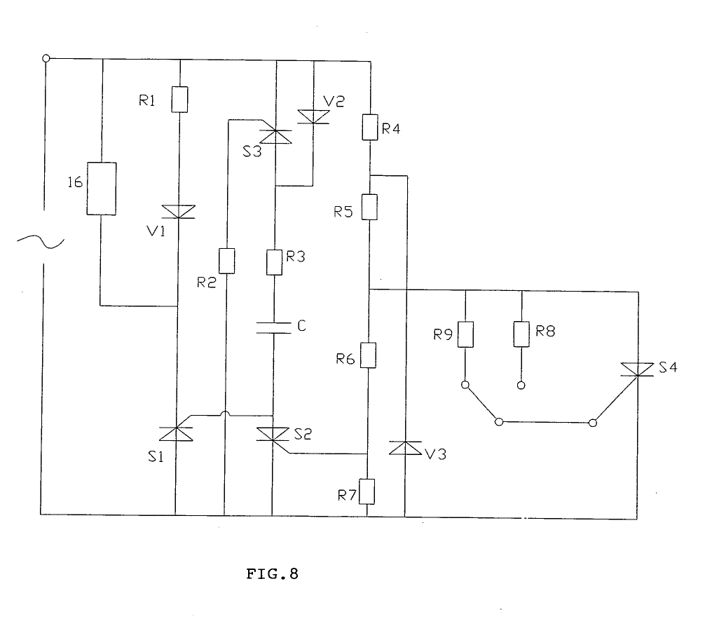

For the described in the FIG. 8, the temperature control circuit is displayed as a layout diagram. The temperature sensors switch is S4. R8, R9 are two initial number set resistors on the high temperature and the low temperature each thereof. In addition, the temperature control circuit can be extended to include R4-R7, and the temperature sensor switch S3 and the capacitor C. Thus the two-direction controllable switch SI in circuit can be formed to execute the temperature control function. But the other related circuit can be applied to use the above similar function.

The process for the temperature controlling can be in the following steps: when the temperature is lower than the related set temperature, the related sensor switch is at the breaking state. Then R4-R7 is generating a related phase signal to activate the sensor switch S3, and at the same period, the two-direction controllable sensor S1 is activated to link the circuit. As the AC current is activated on a positive half period, the capacitor C is charged; as a negative half period current is activated, the capacitor C is discharged. The current activates the switch S1 and assures the positive half period and the negative period both coupling to S1. When the temperature is raised to the set temperature, the switch S4 is linked, and the switch S3 is triggered by a bypass to decouple. The S1 is then decoupling and stopping to supply power to the heater, which allows the natural dissipation to lower down the temperature. When the temperature is lower than the set temperature, the switch S4 is changing from coupling state to decoupling state, which turns S3 and S1 to coupling state to provide power to heat the heater.

The FIG. 7 shows another embodiment of the present invention. The air guiding sleeve 14 has one end connected to the outlet 12 of the casing 13, and another end with contact area to a patient's skin. The energy concentrating element 19 can be installed on the outside range of the air guiding sleeve 14. In addition, the protection frame 24 can be installed on the air guiding sleeve 14 to avoid burning from accidental touch of the air guiding sleeve 14.

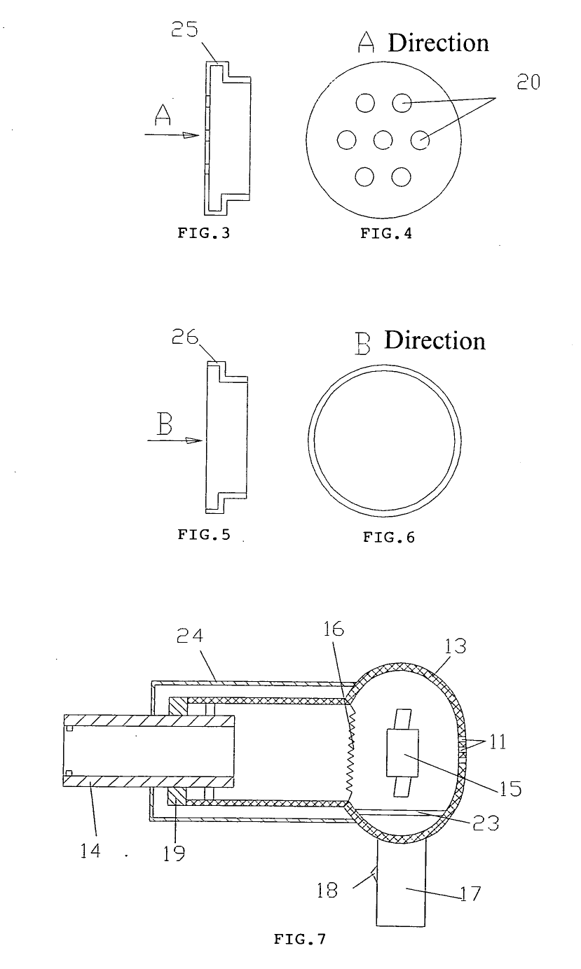

As shown in the FIG. 2, it is another embodiment of the present invention. The air guiding sleeve 14 is further comprising an air guiding frame 25 (FIGS. 3-4); and a plurality of vent holes 20 are uniformly placed on the surface of the air guiding frame 25. The air guiding frame 25 can be made of the material of carbon fiber and metal. The enhanced effect can be achieved by the air guiding frame 25. The air guiding sleeve 14 can be further comprising a frame structure for scraping 26 (FIGS. 5-6). The scraping method can be applied on the skin. And a tube shape is placed on the frame structure for scraping 26 or the end shape of the frame structure for scraping 26 is an arc shape. The frame structure for scraping 26 can be made of the material of carbon fiber and metal. The above described air guiding frame 25 can be structurally interchangeable with the frame structure for scraping 26.

Although the invention has been described and illustrated with reference to specific illustrative embodiments thereof, it is not intended that the invention is limited to those illustrative embodiments. Those skilled in the art will recognize that variations and modifications can be made without departing from the spirit of the invention. It is therefore intended to include within the invention all such variations and modifications which fall within the scope of the appended claims and equivalents thereof. One skilled in the art will understand that the embodiment of the present invention as shown in the drawings and described above is exemplary only and not intended to be limiting.

The foregoing description of the preferred embodiment of the present invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

What is claimed is:

1. A health care and physical therapy device for gathering energy, comprising:
a casing, said casing having an inlet and an outlet on two opposite sides thereof;
a fan, said fan installed inside the casing;
a heater, said heater installed in the casing;
an air guiding sleeve, said air guiding sleeve having one end connected to the outlet of the casing and another end with contact area to a patient's skin, said air guiding sleeve having an exhaust opening; and
an energy concentrating element, said energy concentrating element installed in said air guiding sleeve.

2. The health care and physical therapy device for gathering energy according to claim 1, further comprising:
a temperature sensor installed on said air guiding sleeve on one end near the contact area of patient's skin; and
a temperature control circuit connecting to said temperature sensor.

3. The health care and physical therapy device for gathering energy according to claim 2, wherein said temperature control circuit connected to said heater and said fan, whereby based on the measured temperature by said temperature sensor, said temperature control circuit modulates the power of said fan and said heater.

4. The health care and physical therapy device for gathering energy according to claim 1, wherein an exhaust opening is installed on one place selected from the structure of the side wall of said air guiding sleeve, or on the connecting portion of said air guiding sleeve with said outlet of said casing.

5. The health care and physical therapy device for gathering energy according to claim 1, wherein said air guiding sleeve has at least one vent hole on the near side of the contact area to patient's skin.

6. The health care and physical therapy device for gathering energy according to claim 1, wherein said energy concentrating element is a circle structure made of the material selected from the group of copper, silver, and carbon fiber.

7. The health care and physical therapy device for gathering energy according to claim 1, wherein said heater is made of the material selected from the group of heating wire, heating tube, and infrared tube.

8. The health care and physical therapy device for gathering energy according to claim 1, wherein said casing further comprising a handle for operation; and a switch on said handle.

9. The health care and physical therapy device for gathering energy according to claim 1, wherein said air guiding sleeve further comprising an air guiding frame; and a plurality of vent holes evenly spread on the surface of said air guiding frame.

10. The health care and physical therapy device for gathering energy according to claim 1, wherein said air guiding sleeve further comprising a frame structure for scraping; and a tube shape is placed on the frame structure for scraping.
